(19) United States
(12) Patent Application Publication
(10) Pub. No.: US 2014/0336556 A1
(43) Pub. Date: Nov. 13, 2014

(54) POSTURE SUPPORT GARMENT

(71) Applicant: TheraChic LLC, Los Gatos, CA (US)

(72) Inventor: Sophia Pucik, Los Gatos, CA (US)

(73) Assignee: TheraChic LLC, Los Gatos, CA (US)

(21) Appl. No.: 14/276,928

(22) Filed: May 13, 2014

Related U.S. Application Data
(60) Provisional application No. 61/822,522, filed on May 13, 2013.

(51) Int. Cl. A61F 5/02

(52) U.S. Cl. CPC A61F 5/02
USPC 602/19; 2/69

(57) ABSTRACT

Disclosed is a posture support garment. The posture support garment can be designed to use an elastic tension fabric that provides tension on the left sleeve and right sleeve of the posture support garment. The tension created can pull the back of the left sleeve and the right sleeve inwards towards the middle of the wearer of the posture support garment's back. This, in turn, can cause the wearer's shoulders to be pulled back and upwards, thus stretching the wearer's chest and shoulder muscles while also relieving tension on the wearer's back and neck muscles. This can help reverse the effects of repetitive poor posture as well as the physical pains associated with poor posture. The elastic tension fabric can be concealed beneath a back panel to the posture support garment which allows the elastic tension fabric to stretch and contract without affecting the appearance of the back panel.

### CROSS REFERENCE TO RELATED APPLICATIONS

This application claims priority to U.S. provisional application No. 61/822,522, filed on May 13, 2013, which is expressly incorporated by reference herein in its entirety.

### TECHNICAL FIELD

The present disclosure relates to posture aids and more specifically to an adjustable posture support garment.

### BACKGROUND

Poor posture can lead to a variety of bodily pains, including neck and back pain. Poor posture often results in certain muscles tightening up or shortening while others lengthen and become weak. This may be the result of repetitive motion resulting from long work hours. For example, sitting at a desk hunched over can cause the chest and shoulder muscles to remain in a contracted short state causing the muscles to tighten, while at the same time, the back muscles are lengthened and become weak. Extended periods of poor posture can cause the chest and shoulder muscles to get so tight and the back muscles to get so weak that the body begins to naturally position itself with poor posture.

To correct the effects of repetitive poor posture and the associated neck and back pain, proper posture must be practiced for extended periods of time. While positioning the body to use correct posture may be easy, holding this position at all times may be hard. For example, when working the body will naturally revert to the poor posture position. Thus, without a persistent reminder a person can easily revert back to poor posture without even noticing.

Common solutions include rigid braces that hold the shoulders in a proper position. While these types of braces can be effective, they often provide the wearer with a reduced range of motion. Further, these types of braces are often bulky, uncomfortable and cumbersome and cannot be worn undetected. Thus, a person may be less likely to wear it at all times. Accordingly, there is a need for an improved posture aid.

### SUMMARY

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims, or can be learned by the practice of the principles set forth herein.

Disclosed is an adjustable posture support garment. The adjustable posture support garment can be designed to use an elastic tension fabric that provides tension to the top portion of the left sleeve and the top portion of the right sleeve of the posture support garment, which pulls the left sleeve and the right sleeve inwards towards the middle of the wearer of the posture support garment's back. This in turn pulls the wearer's shoulders back and assists in positioning the shoulders and arms in a position promoting good posture.

The elastic tension fabric can be attached to the left and right sleeves of the posture support garment directly onto the sleeves and at the front and/or back seam(s) of the left sleeve and the front and/or back seam(s) of the right sleeve, causing the elastic tension fabric to wrap over the wearer's left and right shoulders at the left and right sleeves and create tension that pulls the wearers shoulders back and towards each other. The tension in the sleeves serves as an aid and reminder for the wearer to position shoulders and arms correctly to support good posture.

To conceal the elastic tension fabric and thus conceal that the wearer is wearing a posture support garment, the posture support garment can include a back panel that provides a back to the posture support garment and also covers the elastic tension fabric. To allow the elastic tension fabric to move freely without substantially affecting the appearance of the back panel, the back panel and the elastic tension fabric can be independent of each other, meaning that the back panel and elastic tension fabric are not directly attached. Further, the posture support garment can be designed such that the back panel is not attached to the back of the left and right sleeve along the back seam of the left and right sleeve. This can further allow the elastic tension fabric and the right and left sleeve to move independently without the stretching and contracting of the elastic tension fabric directly affecting the shape of the back panel. The appearance of the back panel of therefore remains substantially unaffected by the stretching and contracting of the elastic tension fabric.

In some embodiments, the elastic tension fabric can be adjustable to allow the wearer to adjust the tension pulling back the wearer's shoulders. For example, the elastic tension fabric can be split into a right portion and a left portion and use one of various adjustable connecting methods to connect the two portions. For example, the left and right portion can be connected using a lace, hook and loop, hook and eye, fastener element, snaps, pins, clasps, Velcro®, etc. The length of the elastic tension fabric can be adjusted using the provided connecting method.

### BRIEF DESCRIPTION OF THE DRAWINGS

The above-recited and other advantages and features of the disclosure will become apparent by reference to specific embodiments thereof which are illustrated in the appended drawings. Understanding that these drawings depict only exemplary embodiments of the disclosure and are not therefore to be considered to be limiting of its scope, the principles herein are described and explained with additional specificity and detail through the use of the accompanying drawings in which:

FIGS. 1A and 1B illustrates an exemplary embodiment of a posture support garment

FIGS. 2A-2C illustrate exemplary embodiments of a posture support garment with an adjustable elastic tension fabric;

FIG. 3 illustrates another exemplary embodiment of an adjustable elastic tension fabric;

FIG. 4 illustrates another exemplary embodiment of an adjustable elastic tension fabric;

FIG. 5 illustrates an embodiment of posture support garment designed such that the elastic tension fabric the crosses the wearer's back as two separate straps;

FIG. 6 illustrates an embodiment of a posture support garment where the elastic tension fabric connects along the back of the posture support garment to create a unified elastic tension fabric;

FIG. 7A-7C illustrate exemplary embodiments of a posture support garment including a placement element;

FIG. 8 illustrates an exemplary embodiment of a posture support garment including a front fastening element; and

FIGS. 9A and 9B illustrate an exemplary embodiment of a posture support garment where the tension fabric is woven into the posture support garment.

### DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure.

The present disclosure addresses the need in the art for an improved posture support garment. Traditional posture braces utilize rigid and bulky straps that physically hold the wearer's shoulders in a specified position. While effective at holding the shoulders in a proper position, these types of posture braces are not practical for daily use. For example, the rigid and bulky design limits the wearer's range of motion. This not only affects the wearer's ability to perform routine tasks, but also brings unwanted attention to the fact that the wearer is wearing a posture brace. In addition to limiting motion, the bulky design cannot be easily hidden and may be difficult to put on and remove, resulting in a user choosing to not wear the posture brace.

The present disclosure discloses an adjustable posture support garment that can be worn undetected while providing adequate tension to aid in the correction of the wearer's posture. Additionally, the posture support garment can provide the user with a full range of motion and is simple to put on and remove. To accomplish this, the posture support garment can include an elastic tension fabric connecting the left and right sleeves of the posture support garment wherein the tension created by the elastic tension fabric pulls the left and right sleeve of the posture support garment towards the middle of the wearer's back, causing the wearer's shoulders and arms to be pulled into a position promoting good posture. The elastic tension fabric can be hidden beneath a back panel of the posture support garment that provides a back to the posture support garment and conceals the pullback of the sleeves and elastic tension fabric.

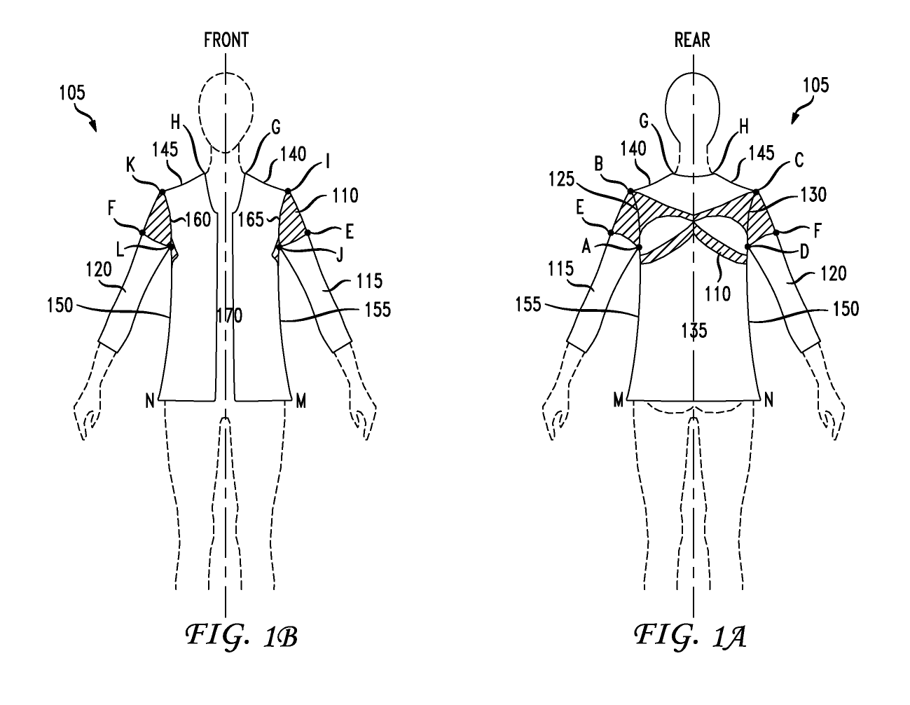

FIGS. 1A and 1B illustrate a front and back view of an exemplary embodiment of a posture support garment. As shown elastic tension fabric 110 connects to right sleeve 120 at seam 160 illustrated in FIG. 1B as a line running from point L to point K. Further elastic tension fabric connects to left sleeve 115 at seam 165 illustrated in FIG. 1 B as a line running from point I to J. Elastic tension fabric 110 can be positioned beneath left sleeve 115 and right sleeve 120, thereby concealing the fact that posture support garment 105 includes elastic tension fabric 110 below left sleeve 115 and right sleeve 120.

As shown, elastic tension fabric 110 runs from seam 160 along the top of the right sleeve 120 to the middle of the posture support garment wearer's back. Likewise, elastic tension fabric 110 runs from seam 165 along the top of the left sleeve 115 to the middle of the posture support garment wearer's back. Elastic tension strap 110 creates tension between left sleeve 115 and right sleeve 120, thereby pulling a wearer's shoulders backwards.

As shown, elastic tension fabric 110 can continue from seam 160 on right sleeve 120 and seam 165 on left sleeve 115 to below the wearer's armpit on each arm and connect behind the posture support garment wearer's back. This creates tension between left sleeve 115 and right sleeve 120 that pulls the wearer's shoulders back and upwards.

FIG. 1A shows a rear view of the posture support garment. As shown, posture support garment 105 includes elastic tension fabric 110, which connects to the top 1 to 4 inches of left sleeve 115 to the top 1 to 4 inches of right sleeve 120. Posture support garment 105 can further include back panel 135 that conceals elastic tension fabric 110. Elastic tension fabric 110 is thus hidden by back panel 135 and not visible when viewing the back of posture support garment 105. Areas of posture support garment 105 illustrated in FIG. 1A that are concealed by back panel 135 and left sleeve 115 and right sleeve 120 are filled with diagonal stripes.

Elastic tension fabric 110 can additionally be attached to left sleeve 115 at seam 125, which is illustrated as the line running from point A to point B in FIG. 1A. Further, elastic tension fabric 110 can additionally be attached to right sleeve 120 at seam 130, which is illustrated as a line running from point C to point D in FIG. 1A. Elastic tension fabric 110 can be attached to left sleeve 115 and right sleeve 125 using any of a variety of known methods in the art, such as sewing, stitching, etc.

Elastic tension fabric 110 can provide tension to left sleeve 115 and right sleeve 120, causing left sleeve 115 and right sleeve 120 to be pulled back and towards each other. The tension created by elastic tension fabric 110, combined with tension created by left sleeve 115 and left sleeve 120 on the wearer's shoulders, causes the wearer's shoulders to be pulled back, thus elongating the wearer's chest and shoulder muscles while relieving tension on the wearer's upper back muscles. Specifically, posture support garment 105 stretches and elongates the wearer's deltoid, pectoralis major and pectoralis minor muscles while releasing pressure on the wearer's supraspinatus, infraspinatus, rhomboids and lattisimus dorsi muscles, thus aiding the wearer to achieve good posture.

To accomplish this, elastic tension fabric 110 can be made of an elastic material suitable to provide adequate tension to pull back the wearer's shoulders. For example, in some embodiments, elastic tension fabric 110 can be made of a stretch fabric such as spandex or elastane. Using a stretch fabric such as spandex or elastane can provide adequate tension to properly pull back the wearer's shoulders, thus encouraging correct posture. Further, the elastic properties of a stretch fabric can also provide the wearer with a full range of motion when necessary.

The tension created by elastic tension fabric 110 can be variable based on the length of elastic tension fabric 110 and the elasticity of the material used from elastic tension fabric 110. For example, the shorter the length of elastic tension fabric 110, the greater the tension on left sleeve 115 and right sleeve 120. Likewise, the longer the length of elastic tension fabric 110, the weaker the tension on left sleeve 115 and right sleeve 120. Further, the greater the elasticity of the material used for elastic tension fabric 110, the weaker the tension on left sleeve 115 and right sleeve 120. Likewise, the lower the elasticity of the material used for elastic tension fabric 110, the greater the tension of left sleeve 115 and right sleeve 120.

In some embodiments, the length of elastic tension fabric 110 can be directly proportional to the elasticity of the material used to create elastic tension fabric 110. For example, the length of elastic tension fabric 110 can be shorter when the material used is highly elastic, thus resulting in adequate force on the wearer's shoulders. Likewise, to create adequate force of the wearer's shoulders, the length of elastic tension fabric 110 can be longer when the material used has low elasticity.

To conceal elastic tension fabric 110, in some embodiments, posture support garment 105 can include back panel 135 that covers the wearer's back and is placed over elastic tension fabric 110. Thus, back panel 135 can conceal the entirety of elastic tension fabric 110.

In some embodiments, back panel 135 can be independent of elastic tension fabric 110. In this type of embodiment, elastic tension fabric 110 is only attached at front seams 160 and 165 illustrated in FIG. 1B as well as along part of the top portion of the sleeve 115 between points J to E and the top portion of sleeve 120 between points L to F. Although, in the above described embodiment, back seam 125 of left sleeve 115 and back seam 130 of right sleeve 120 are not attached to elastic tension fabric 110, this does not mean that elastic tension fabric cannot be attached to these seams in other embodiments. In some embodiments, elastic tension fabric 110 can be thinner than the length of seam 125 and seam 130, and thus a portion of both seam 125 and seam 130 can be attached to back panel 135 and not attached to elastic tension fabric 110.

Back panel 135 can be attached to front panel 170 (shown in FIG. 1B) of posture support garment 105 at seam 140 running from point B to point G, seam 145 running from point C to point H, seam 125 running from point B to A, seam 130 running from point C to D, seam 150 running from point A to point M and seam 155 running from point D to point N.

FIG. 1B shows a front view of posture support garment 105. As shown, posture support garment 105 can include front panel 170. Front panel 170 can be attached to back panel 135 (shown in FIG. 1A) at seams 140, 145, 150 and 155. Further, front panel 170 can be connected to left sleeve 115 at seam 165 running from point I to point J, and right sleeve 120 at seam 160 running from point K to point L.

Returning to the discussion of FIG. 1A, in some embodiments, the force created by posture support garment 105 to pull back a wearer's shoulders can be set based on the length of elastic tension fabric 110. For example, the shorter elastic tension fabric 110 is, the greater the force created on left sleeve 115 and right sleeve 120. In some embodiments, elastic tension fabric 110 can be a set length such that it cannot be adjusted.

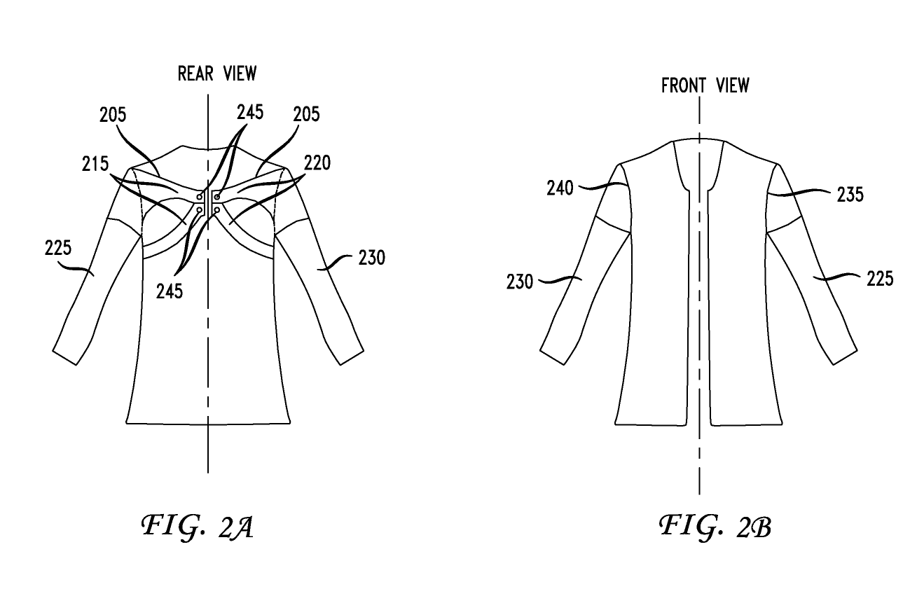

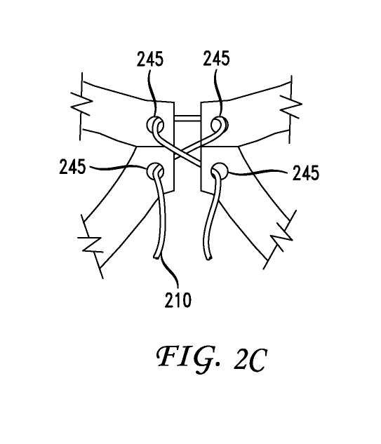

Alternatively, in some embodiments, the length of elastic tension fabric 110 can be adjusted. Thus, a wearer can shorten elastic tension fabric 110 to create greater force pulling back the wearer's shoulders. FIGS. 2A, 2B and 2C illustrate an exemplary embodiment of an adjustable elastic tension fabric.

As shown in FIG. 2A, elastic tension fabric 205 can include lace 205 to adjust the length of elastic tension fabric 205. Elastic tension fabric 205 can include left portion 215 and right portion 220.

As shown in FIG. 2B, left portion 215 can be attached to left sleeve 225 at seam 235 and right portion 220 can be attached to right sleeve 230 at seam 240. Further, left portion 215 and right portion 220 can include lace holes 245.

As shown in FIG. 2C, lace holes 245 can be big enough to allow lace 210 to be strung through lace holes 245. Opposite ends of lace 210 can be tied together to maintain a desired length of elastic tension fabric 205. Thus, the length of elastic tension fabric 205 can be adjusted by loosening or tightening lace 210 through lace holes 245 to create a smaller or larger distance between left portion 215 and right portion 220. Opposite ends of lace 210 can be tied together in a knot to maintain the selected length of elastic tension fabric 205.

Although only two columns of lace holes 245 are shown per left portion 215 and right portion 220, this is only one embodiment and is not meant to be limiting. One skilled in the art would recognize that right portion 215 and left portion 220 of elastic tension fabric 205 can each include any number of columns of lace holes 245, and this disclose recognizes all such embodiments.

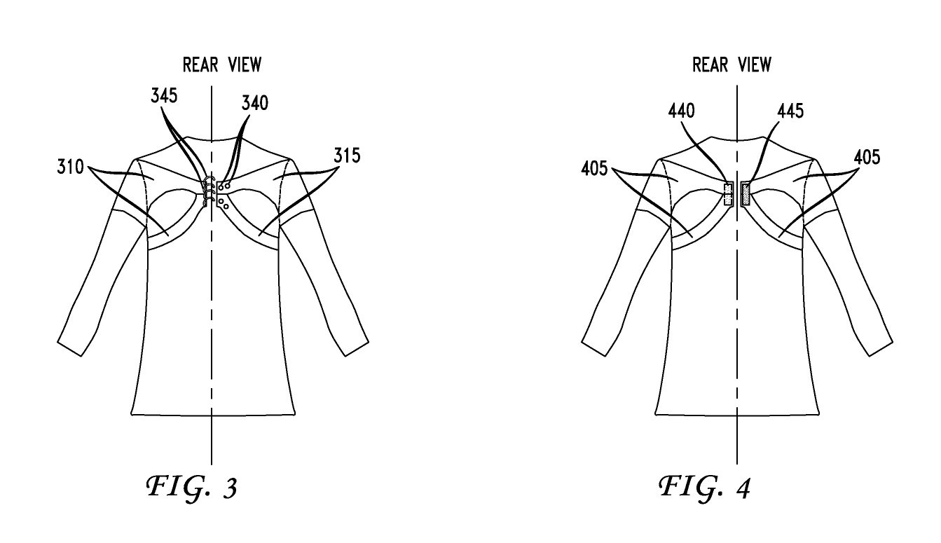

FIG. 3 illustrates another exemplary embodiment of an adjustable elastic tension fabric. As shown, elastic tension fabric 305, includes left portion 310 and right portion 315. As shown, right portion 315 can include multiple columns of eyes 340. Eyes 340 can be connected to left portion 310 so that at least one side of each of eyes 340 can be lifted from left portion 310. Right portion 315 can include hooks 345 that can be used to fasten to eyes 340 and thus connect left portion 310 to right portion 315. The length of elastic strap 305 can be adjusted by changing the column of eyes 340 to which hooks 345 are fastened. In some embodiments, hooks 345 can be S hooks.

FIG. 4 illustrates another exemplary embodiment of an adjustable elastic tension fabric. As shown elastic strap 405 includes left portion 410 and right portion 415. As shown left portion 405 includes fastener element 440 and right portion 405 includes fastener element 445. Fastener element 440 and 445 can be a hook and loop type fastener such as Velcro®. As shown, fastener element 445 can be on the back side of right strap such that fastener element 445 can be connected to fastener element 440 by overlapping left portion and right portion. The length of elastic tension fabric 405 can thus be adjusted by changing the position at which left portion and right portion are connected by fastener element 440 and fastener element 445.

The examples used in FIGS. 2-4 are just some possible embodiments of an adjustable elastic tension fabric. One skilled in the art would recognize that an elastic tension fabric can be adjusted using any of a variety of known techniques in the art, including but not limited to snaps, pins, clasps, etc.

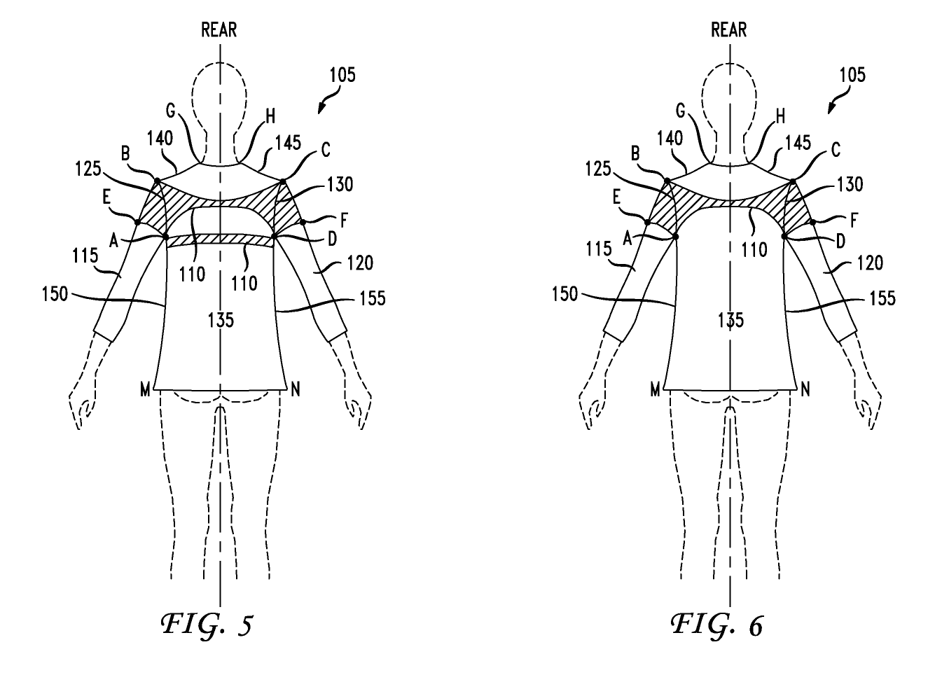

FIG. 5 shown an embodiment of posture support garment 105 designed so that elastic tension fabric 110 crosses the wearer's back as two separate straps. FIG. 6, in contrast, illustrates an embodiment of posture support garment 105 where elastic tension fabric 110 connect along the back of the posture support garment to create a unified elastic tension fabric.

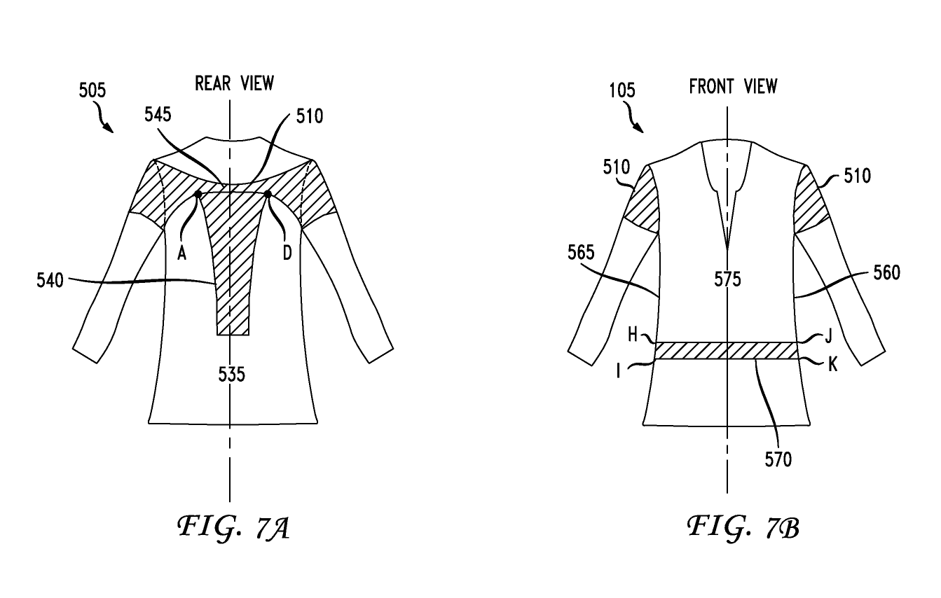

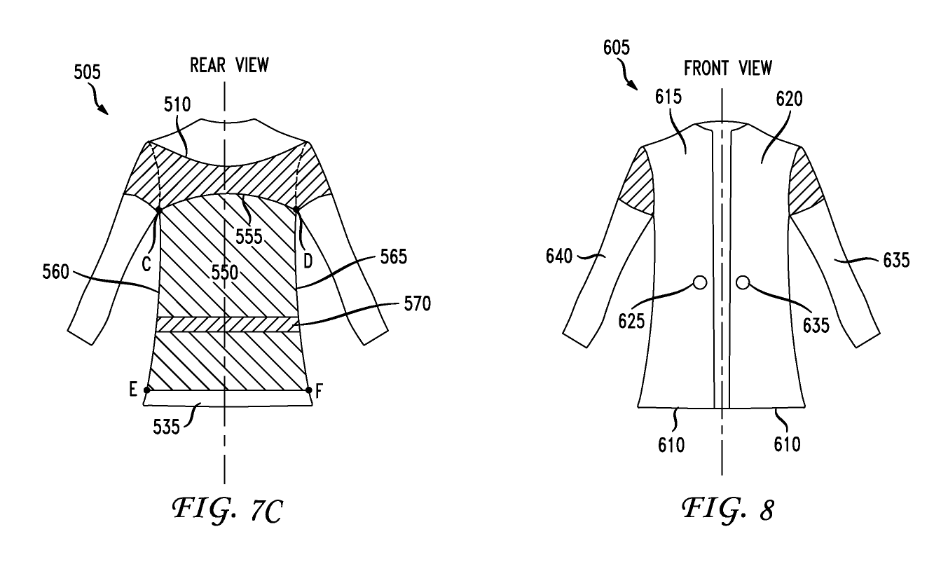

FIGS. 7A-7C illustrate exemplary embodiments of a posture support including a placement element. As shown in FIG. 7A, posture support garment 505 can include elastic tension fabric 510. Further, posture support garment 505 can include back panel 535 that conceals elastic tension fabric 510. Posture support garment 505 can also include placement element 540. Placement element 540 can be attached to elastic tension fabric 510 at seam 545 running from point A to point B. Placement element 540 can be concealed by back panel 535. Further, placement element 540 can be independent of back panel 535, such that placement element 540 is not directly attached to back panel 535 at any point. In some embodiments, placement element 540 and elastic tension fabric 510 can be a single piece rather than two pieces attached at seam 545.

As shown, placement element 540 can hang from elastic tension fabric 510 down to near the bottom of back panel 535. A wearer of posture support garment 505 can thus use their hand to grab and pull placement element 540 to adjust the placement of elastic tension fabric 510 on the wearer's back. For example, if elastic tension fabric 510 begins to move upward along the wearer's back, the wearer can pull downward on placement element 510 to adjust elastic tension fabric 510 downwards along the wearer's back.

The length of placement element 540 can be variable, although it is preferable that placement element 540 be long enough to allow a wearer to easily grab placement element 540, while also being short enough to remain concealed beneath back panel 535. Placement element 540 can be made of any material known in the art. For example, placement element 540 can be made of cotton, polyester, etc.

FIG. 7B illustrates another exemplary embodiment of posture support garment 505 including a placement element to keep elastic tension fabric 510 in a proper or desired position. As shown, posture support garment 505 includes placement element 550 that is much wider than placement element 540 shown in FIG. 5A. In this embodiment, placement element 550 is attached to elastic tension fabric 510 at seam 555 running from point C to point D, and is concealed by back panel 535. Alternatively, in some embodiments, elastic tension fabric 510 and placement element 550 can be a single piece of elasticized fabric rather than two pieces of fabric attached at seam 555.

Placement element 550 can keep elastic tension fabric 510 in the proper position by being attached to posture support garment 505 which anchors elastic tension fabric 510 in the desired or proper positions. For example, as illustrated in FIG. 5B, placement element 550 can be connected to posture support garment 505 at seam 560 running from point C to point E or just a portion of seam 560, and seam 565 running from point D to point F or just a portion of seam 565. Thus, placement element 545 can anchor elastic tension fabric 510 in the correct position while still allowing elastic tension fabric 510 to move freely beneath back panel 535.

In some embodiments, posture support garment 505 can include body strap 570 to further maintain the position of elastic tension fabric 510. Body strap 570 can be attached to placement element 545 along at least a portion of seams 560 and 565. The wearer can position their body between body strap 570 and placement element 545 to add additional support to keep elastic tension fabric 510 in proper placement.

FIG. 7C illustrates a front view of posture support garment 505 including body strap 570. As shown posture support garment 505 includes front panel 575 attached to back panel 535. Body strap 570 can be attached to posture support garment 505 at a portion of seam 565 running from point H to point I, and a portion of seam 560 running from point J to point K. Further, body strap 570 can be concealed beneath front panel 570. In some embodiments, body strap 570 and front panel 570 are not attached at any point other than at seams 565 and 560. A wearer can place body strap 570 in front of their body when wearing posture support garment 505, while elastic tension fabric 510 and placement element 550 are positioned behind the wearer's body. Body strap 570 can thus create tension to further hold elastic tension fabric 510 and placement element 550 in the correct or desired position.

In some embodiments, body strap 570 can be made of an elastic material to create tension around the wearer's body. For example, body strap 570 can be made of a stretch fabric such as spandex or elastane. In some embodiments, body strap 570 can be adjustable. For example, body strap 570 can be designed to incorporate the adjustable features illustrated in FIGS. 2-4 to adjust the elastic tension fabric. Thus, in some embodiments, body strap 570 can incorporate a lace, hook and loop, fastener element, snaps, pins, clasps, etc., to adjust the length of body strap 570.

In some embodiments, the posture support garment can be designed to include a front fastening element that can be used to adjust the tension created on the wearer's left and right shoulders. For example, the posture support garment can be a garment such as a jacket, coat or cardigan that can be opened a closed in the front using a front fastening element such as a button, snap, etc. The wearer can thus adjust the strength of force pulling back the wearer shoulders by opening and/or closing the front of the posture support garment using the front fastening element. For example, to decrease the force pulling back the wearer's shoulders, the wearer can close the front of the posture support garment using either a rigid or an adjustable fastening element. Alternatively, to increase the force pulling the back the wearer's shoulders, the wearer can open the front of the posture support garment using the front fastening element. Further, closing the front of the posture support garment also functions as an anchor by creating tension that encourages the elastic tension fabric to remain in place.

FIG. 8 illustrates an exemplary embodiment of a posture support garment including a front fastening element. A front view of posture support garment 605 is illustrated. As shown, front panel 610 is split into left portion 615 and right portion 620. In some embodiments, left portion 615 and right portion 620 can be connected using a front fastening element. As shown, left element includes front fastening element 625 and right portion 630 includes front fastening element 630 Fastening. Front panel 610 of posture support garment 605 can be closed by connecting left portion 615 to right portion 630 using front fastening element 630.

Front fastening elements 625 and 630 can be any type of fastener known in the art. For example, in some embodiments, front fastening element 625 can be a button and front fastening element 630 can be a button hole designed to receive the button. Alternatively, fastening element 625 and fastening element 630 can be a hook and loop fastener such as Velcro®, or fastening element 625 and fastening element 630 could be a zipper. Although, Velcro®, a zipper and a button are used as examples, this is not meant to be limiting. One skilled in the art would recognize that front fastening elements 625 and 630 can be any of a variety of fasteners known in the art.

Further, in some embodiments, posture support garment 605 can include more front fastening elements than illustrated in FIG. 8. For example, multiple rows and columns of front fastening elements can be included in either or both of left portion 615 and right portion 620 of front panel 610. Thus a wearer could adjust the front panel 610 to adjust the strength of the force on the wearer's shoulders. For example, a wearer can adjust the width of front panel 610 by adjusting the point at which left panel 615 to left panel 620 are connected. By adjusting the width of front panel 610, the wearer can create more or less tension on the wearer's shoulders. For example, the shorter the width front panel 610 is adjusted to using front fastening elements 625 and 620, the less the force created on the wearer's shoulders to pull back the wearer's shoulder and promote good posture. Alternatively, the wearer can adjust the width of front panel 610 to make it wider, and thus increase the force on the wearer's shoulders.

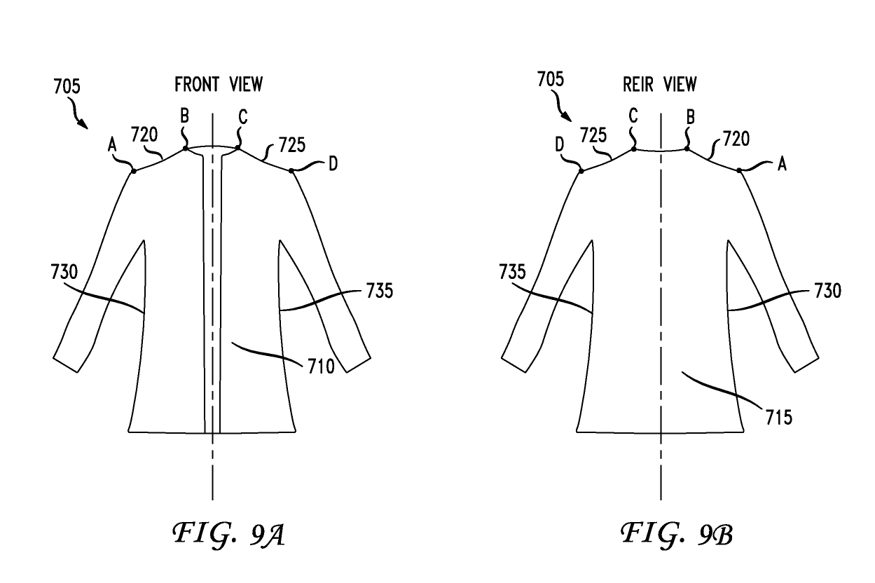

FIGS. 9A and 9B illustrate another exemplary embodiment of a posture support garment. FIG. 9A illustrates a back view of posture support garment 705. As shown, elastic tension fabric 710 of posture support garment 705, is one continuous piece of material that forms the sleeves and back of posture support garment 705. In this embodiment, posture support garment 705 does not include a back panel to conceal elastic tension fabric 710. Rather, concealing the fact that the wearer of posture support garment 705 is wearing a posture support brace is accomplished by designing elastic tension fabric 705 to appear as the back panel of posture support garment 705. Thus elastic tension fabric 710 appears to just be the back panel of a garment rather than a brace providing tension to the wearers shoulder to pull the shoulders back and towards each other. To provide adequate force to the wearer's shoulders to aid in correcting posture, elastic tension support fabric 710 can be can be made of a stretch fabric such as spandex or elastane.

FIG. 9A illustrates a front view of posture support garment 705. As shown, posture support garment 705 can include front panel 715 attached to elastic tension fabric 710. Front panel 715 can be attached to elastic tension fabric 710 at seam 720 running from point A to point B, seam 725 running from point C to point D, seam 735 running from point D to point F, and seam 730 running from point A to point E. Although front panel 715 is illustrated as a continuous panel, this is just one embodiment and is not meant to be limiting. In some embodiments, front panel 715 can consist of a left portion and a right portion that can be attached using an attaching element included on the left portion and the right portion.

The various embodiments described above are provided by way of illustration only and should not be construed to limit the scope of the disclosure. Those skilled in the art will readily recognize various modifications and changes that may be made to the principles described herein without following the example embodiments and applications illustrated and described herein, and without departing from the spirit and scope of the disclosure.

Although the posture support garment has been illustrated as a shirt or jacket throughout this disclosure, this is not meant to be limiting. One skilled in the art would recognize that the posture support garment can be any of a variety of garments, including a shirt, jacket, coat, sweater, blouse, button up shirt, dress, etc.

1. A posture support garment, comprising:
a first elastic tension fabric attached to:
a front of a left sleeve of the posture support garment at a first seam beneath the left sleeve, and
a front of a right sleeve of the posture support garment at a second seam beneath the right sleeve,
wherein, when the posture support garment is worn by a wearer:
the first elastic tension fabric is positioned across a back of the wearer,
the first elastic tension fabric wraps over a left shoulder of the wearer to the front of the left sleeve,
the first elastic tension fabric wraps over a right shoulder of the wearer to the front of the right sleeve, and
the wearer causes the first elastic tension fabric to stretch, thereby creating tension pulling the left shoulder of the wearer and the right shoulder of the wearer inward toward the back of the wearer;
a second elastic tension fabric attached to:
the front of the left sleeve at the first seam, and
the front of the right sleeve at the second seam,
wherein, when the posture support garment is worn by the wearer:
the second elastic tension fabric is positioned across the back of the wearer,
the second elastic tension fabric wraps below the left shoulder of the wearer to front of the left sleeve,
the second elastic tension fabric wraps below the right shoulder of the wearer to front of the right sleeve, and
the wearer causes the second elastic tension fabric to stretch, thereby creating tension pulling the left shoulder of the wearer and the right shoulder of the wearer inward toward the back of the wearer; and
a back panel covering at least a portion of the elastic tension fabric, wherein the back panel is not directly attached to the elastic tension fabric, thereby allowing the elastic tension fabric to stretch and contract beneath the back panel without substantially affecting appearance of the back panel.

2. The posture support garment of claim 1, wherein the first elastic tension fabric and the second elastic tension fabric are one continuous elastic tension fabric.

3. The posture support garment of claim 1, wherein the first elastic tension fabric and the second elastic tension fabric are attached to each other at one or more points between the left sleeve and the right sleeve to create a unified elastic tension fabric.

4. The posture support garment of claim 1, wherein the unified elastic tension fabric comprises a left portion and a right portion, wherein the left portion of the unified elastic tension fabric is detachably coupled to the right portion of the unified elastic tension fabric.

5. The posture support garment of claim 4, wherein:
the left portion of the unified elastic tension fabric comprises at least a first lace hole,
the right portion of the unified elastic tension fabric comprises at least a second lace hole, and
the right portion of the unified elastic tension fabric and the left portion of the unified elastic tension fabric are detachably coupled by a lace that is positioned through the first lace hole and the second hole.

6. The posture support garment of claim 4, wherein the left portion of the unified elastic tension fabric and the right portion of the unified elastic tension fabric are detachably coupled by a hook and loop.

7. The posture support garment of claim 4, wherein:
the left portion of the unified elastic tension fabric comprises a first fastener element,
the right portion of the unified elastic tension fabric comprises a second fastener element, and
the left portion of the unified elastic tension fabric and the right portion of the unified elastic tension fabric are detachably coupled by the first fastener element and the second fastener element.

8. The posture support garment of claim 3, further comprising:
a placement element attached to the unified elastic tension fabric, wherein the placement element hangs down from the unified elastic tension fabric, thereby allowing the wearer to use the placement element to adjust placement of the unified elastic tension fabric along the wearer's back.

9. The posture support garment of claim 8, further comprising:
a body strap including a first end and a second end, wherein the body strap is attached to the placement element at the first end and the second end, allowing the wearer to position the body strap around their body while wearing the posture support garment.

10. A method of wearing a posture support garment, comprising:
positioning, by a wearer of the posture support garment, a first arm through a first sleeve of the posture support garment, wherein:
a front of the first sleeve of the posture support garment is attached to a first elastic tension fabric at a first seam beneath the first sleeve,
the front of the first sleeve of the posture support garment is attached to a second elastic tension fabric at the first seam beneath the first sleeve,
a front of a second sleeve of the posture support garment is attached to the first elastic tension fabric at a second seam beneath the second sleeve,
the front of the second sleeve of the posture support garment is attached to the second elastic tension fabric at the second seam beneath the second sleeve, and
at least a portion of the first elastic tension fabric is concealed by a back panel of the posture support garment; and
positioning, by the wearer of the posture support garment, a second arm through the second sleeve of the posture support garment, wherein the first elastic tension fabric:
is positioned across a back of the wearer,
wraps over a first shoulder of the wearer, and
wraps over a second shoulder of the wearer,
thereby causing the first elastic tension fabric and the second elastic tension fabric to stretch and create tension pulling inward from the first sleeve and the second sleeve.

11. The method of claim 10, wherein the back panel is not directly attached to the elastic tension fabric, the back of the right sleeve or the back of the left sleeve, thereby allowing the elastic tension fabric to stretch and contract beneath the back panel without substantially affecting appearance of the back panel.

12. The method of claim 10, wherein the first elastic tension fabric and the second elastic tension fabric are attached to each other at one or more points between the first sleeve and the second sleeve to create a unified elastic tension fabric.

13. The method of claim 12, further comprising:
coupling a first portion of the unified elastic tension fabric to a second portion of the unified elastic tension fabric, resulting in the unified elastic tension fabric being a first length and creating tension of a first level when stretched.

14. The method of claim 13, further comprising:
uncoupling the first portion of the unified elastic tension fabric from the second portion of the unified elastic tension fabric; and
recoupling the first portion of the unified elastic tension fabric to the second portion of the unified elastic tension fabric, resulting in the unified elastic tension fabric being a second length, different than the first length, and creating tension of a second level, different than the first level, when stretched.

15. A method of building a posture support garment, comprising:
attaching a first elastic tension fabric to:
a front of a left sleeve of the posture support garment at a first seam beneath the left sleeve, and
a front of a right sleeve of the posture support garment at a second seam beneath the right sleeve,
wherein, when the posture support garment is worn by a wearer:
the first elastic tension fabric is positioned along a back of the wearer,
the first elastic tension fabric wraps over a right shoulder of wearer and a left shoulder of the wearer, and
the wearer causes the first elastic tension fabric to stretch, thereby creating a tension pulling the right shoulder of the wearer and the left shoulder of the wearer towards each other; and
attaching a back panel of the posture support garment that covers at least a portion of the first elastic tension fabric, wherein the back panel is not directly attached to the first elastic tension fabric, thereby allowing the elastic tension fabric to stretch and contract beneath the back panel without substantially affecting appearance of the back panel.

16. The method of claim 15, further comprising:
attaching a second elastic tension fabric to:
the front of the left sleeve of the posture support garment at the first seam beneath the left sleeve, and
the front of the right sleeve of the posture support garment at the second seam beneath the right sleeve,
wherein, when the posture support garment is worn by a wearer:
the second elastic tension fabric is positioned along a back of the wearer,
the second elastic tension fabric wraps under a right armpit of wearer and a left armpit of the wearer, and
the wearer causes the second elastic tension fabric to stretch, thereby creating a tension pulling the right shoulder of the wearer and the left shoulder of the wearer towards each other.

17. The method of claim 15, wherein the first elastic tension fabric and the second elastic tension fabric are attached to each other at one or more points between the left sleeve and the right sleeve to create a unified elastic tension fabric.

18. The method of claim 17, wherein the unified elastic tension fabric comprises a left portion and a right portion, wherein the left portion of the unified elastic tension fabric can be detachably coupled to the right portion of the unified elastic tension fabric.

19. The method of claim 18, further comprising:
attaching a first fastener element to the left portion of the unified elastic tension fabric; and
attaching a second fastener element to the right portion of the unified elastic tension fabric, wherein the left portion of the unified elastic tension fabric and the right portion of the unified elastic tension fabric can be detachably coupled using the first fastener element and the second fastener element.

20. The method of claim 17, further comprising:
attaching a placement element to the unified elastic tension fabric, wherein the placement element hangs down from the unified elastic tension fabric, thereby allowing the wearer to use the placement element to adjust placement of the unified elastic tension fabric along the wearer's back.
